(19) United States
(12) Patent Application Publication
(10) Pub. No.: US 2006/0240105 A1
(43) Pub. Date: Oct. 26, 2006

(54) Multiparticulate modified release composition

(75) Inventors: John G. Devane, Athlone (IE); Paul Stark, Glassoh (IE); Niall M. M. Fanning, Athlone (IE); Gurvinder Singh Rekhi, Suwanee, GA (US); Scott A. Jenkins, Downingtown, PA (US); Gary Liversidge, Westchester, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Elan Corporation, plc, Dublin (IE)

(21) Appl. No.: 11/372,857

(22) Filed: Mar. 10, 2006

Related U.S. Application Data
(63) Continuation-in-part of application No. 10/827,689, filed on Apr. 19, 2004.
Application No. 10/827,689 is a continuation of application No. 10/354,483, filed on Jan. 30, 2003, now Pat. No. 6,793,936.
Application No. 10/354,483 is a continuation of application No. 10/331,754, filed on Dec. 30, 2002, now Pat. No. 6,902,742.
Application No. 10/331,754 is a continuation of application No. 09/850,425, filed on May 7, 2001, now Pat. No. 6,730,325.
Application No. 09/850,425 is a continuation of application No. 09/566,636, filed on May 8, 2000, now Pat. No. 6,228,398.
Application No. 09/566,636 is a continuation of application No. PCT/US99/25632, filed on Nov. 1, 1999.
(60) Provisional application No. 60/106,726, filed on Nov. 2, 1998.

(51) Int. Cl. A61K 31/485; A61K 31/192; A61K 9/26

(52) U.S. Cl. 424/470; 514/282; 514/570

(57) ABSTRACT

The invention relates to a multiparticulate modified release composition that, upon administration to a patient, delivers at least one active ingredient in a bimodal or multimodal manner. The multiparticulate modified release composition comprises a first component and at least one subsequent component; the first component comprising a first population of active ingredient containing particles and the at least one subsequent component comprising a second population of active ingredient containing particles wherein the combination of the components exhibit a bimodal or multimodal release profile. The invention also relates to a solid oral dosage form containing such a multiparticulate modified release composition.

### CROSS-REFERENCE TO RELATED APPLICATIONS

This is a continuation-in-part of application Ser. No. 10/827,689, filed Apr. 19, 2004, which is a continuation of application Ser. No. 10/354,483, filed Jan. 30, 2003, now U.S. Pat. No. 6,793,936, which in turn is a continuation of application Ser. No. 10/331,754, filed Dec. 30, 2002, now U.S. Pat. No. 6,902,742, which in turn is a continuation of application Ser. No. 09/850,425, filed May 7, 2001, now U.S. Pat. No. 6,730,325, which in turn is a continuation of application Ser. No. 09/566,636, filed May 8, 2000, now U.S. Pat. No. 6,228,398, which in turn is a continuation of Application No. PCT/US99/25632, filed Nov. 1, 1999, which claims the benefit of provisional Application No. 60/106,726, filed Nov. 2, 1998.

### FIELD OF THE INVENTION

The present invention relates to multiparticulate modified release compositions. In particular the present invention relates to multiparticulate modified release compositions that in operation deliver one or more active ingredients in a bimodal or multimodal manner. The present invention further relates to solid oral dosage forms containing such multiparticulate controlled release compositions as well as methods for delivering one or more active ingredients to a patient in a bimodal or multimodal manner.

### DESCRIPTION OF THE PRIOR ART

The effectiveness of pharmaceutical compounds in the prevention and treatment of disease states depends on a variety of factors including the rate and duration of delivery of the compound from the dosage form to the patient. The combination of delivery rate and duration exhibited by a given dosage form in a patient can be described as its in vivo release profile and, depending on the pharmaceutical compound administered, will be associated with a concentration and duration of the pharmaceutical compound in the blood plasma, referred to as a plasma profile. As pharmaceutical compounds vary in their pharmacokinetic properties such as bioavailability, and rates of absorption and elimination, the release profile and the resultant plasma profile become important elements to consider in designing effective drug therapies.

The release profiles of dosage forms may exhibit different rates and durations of release and may be continuous or pulsatile. Continuous release profiles include release profiles in which one or more pharmaceutical compounds are released continuously, either at a constant or variable rate, and pulsatile release profiles include release profiles in which at least two discrete quantities of one or more pharmaceutical compounds are released at different rates and/or over different time frames. For any given pharmaceutical compound or combination of such compounds, the release profile for a given dosage form gives rise to an associated plasma profile in a patient. Similar to the variables applicable to the release profile, the associated plasma profile in a patient may exhibit constant or variable blood plasma concentration levels of the pharmaceutical compounds in the dosage form over the duration of action and may be continuous or pulsatile. Continuous plasma profiles include plasma profiles of all rates and duration which exhibit a single plasma concentration maximum. Pulsatile plasma profiles include plasma profiles in which at least two higher blood plasma concentration levels of pharmaceutical compound are separated by a lower blood plasma concentration level. Pulsatile plasma profiles exhibiting two peaks may be described as “bimodal.”

When two or more components of a dosage form have different release profiles, the release profile of the dosage form as a whole is a combination of the individual release profiles. The release profile of a two-component dosage form in which each component has a different release profile may described as “bimodal.” For dosage forms of more than two components in which each component has a different release profile, the resultant release profile of the dosage form may be described as “multimodal.” Depending on, at least in part, the pharmacokinetics of the pharmaceutical compounds that are used as well as the specific release profiles of the components of the dosage form, a bimodal or multimodal release profile may result in either a continuous or a pulsatile plasma profile in a patient.

Conventional frequent dosage regimes in which an immediate release (IR) dosage form is administered at periodic intervals typically gives rise to a pulsatile plasma profile. In such cases, a peak in the plasma drug concentration is observed after administration of each IR dose with troughs (regions of low drug concentration) developing between consecutive administration time points. Such dosage regimes (and their resultant pulsatile plasma profiles) can have particular pharmacological and therapeutic effects associated with them that are beneficial for certain drug therapies. For example, the wash out period provided by the fall off of the plasma concentration of the active ingredient between peaks has been thought to be a contributing factor in reducing or preventing patient tolerance to various types of drugs.

Many controlled release drug formulations are aimed at producing a zero-order release of the drug compound. Indeed, it is often a specific object of these formulations to minimize the peak-to-trough variation in plasma concentration levels associated with conventional frequent dosage regimes. For certain drugs, however, some of the therapeutic and pharmacological effects intrinsic in a pulsatile system may be lost or diminished as a result of the constant or nearly constant plasma concentration levels achieved by zero-order release drug delivery systems. Thus, modified release compositions or formulations which substantially mimic the release of frequent IR dosage regimes, while reducing the need for frequent dosing, is desirable. Similarly, modified release compositions or formulations which combine the benefits of at least two different release profiles to achieve a resultant plasma profile exhibiting pharmacokinetic values within therapeutically effective parameters is also desirable.

A typical example of a drug which may produce tolerance in patients is methylphenidate. Methylphenidate, or α-phenyl-2-piperidine acetic acid methyl ester, is a stimulant affecting the central nervous and respiratory systems and is primarily used in the treatment of attention deficit hyperactivity disorder (ADHD). After absorption from the gastrointestinal tract (GIT), drug effects persist for 3-6 hours after oral administration of conventional IR tablets or up to about 8 hours after oral administration of extended release formulations. The total dosage is typically in the range of 5-30 mg per day, in exceptional cases rising to 60 mg/day. Under conventional dosage regimes, methylphenidate is given twice daily, typically with one dose given before breakfast and a second dose given before lunch. The last daily dose is preferably given several hours before retiring. Adverse effects associated with methylphenidate treatment include insomnia and the development of patient tolerance.

WO 98/14168 (Alza Corp.) teaches a dosage form and a method of administering methylphenidate in a sustained and constantly ascending rate. The dosage form disclosed comprises a plurality of beads comprising a hydrogel matrix with increasing amounts of the active ingredient therein, coated with varying amounts of a release rate controlling material. Appropriate combinations of the active ingredient dose and the number and thickness coating layers can be selected to give an ascending release profile in which the plasma concentration of the active ingredient continually increases over a given period of time. An object of WO 98/14168 is to release a dosage form at a constantly ascending rate specifically to avoid uneven blood levels (characterized by peaks and troughs) associated with conventional treatments using immediate release dosage formulations. As a result, this formulation does not deliver the active ingredient in either a pulsatile or a bimodal manner.

WO 97/03672 (Chiroscience Ltd.) discloses that methylphenidate exhibits a therapeutic effect when administered in the form of a racemic mixture or in the form of a single isomer (such as the RR d-threo enantiomer). Further, WO 97/03763 (Chiroscience Ltd.) discloses a sustained release formulation containing d-threo methylphenidate (dtmp). This disclosure teaches the use of a composition comprising a coating through which the dtmp passes in order to attain sustained release and achieve serum levels (of the active ingredient) of at least 50% cₘₐₓ over a period of at least 8 hours. As above, this formulation does not deliver the active ingredient in either a pulsatile or a bimodal manner.

Shah et al., J Cont. Rel. (1989) 9:169-175 purports to disclose that certain types of hydroxypropyl methylcellulose ethers compressed into a solid dosage form with a therapeutic agent may produce a bimodal release profile. However, it is noted that while polymers from one supplier yielded a bimodal profile, the same polymers with almost identical product specifications obtained from a different source gave non-bimodal release profiles.

Giunchedi et al., Int. J. Pharm (1991) 77:177-181 discloses the use of a hydrophilic matrix multiple-unit formulation for the pulsed release of ketoprofen. Giunchedi et al. teach that ketoprofen is rapidly eliminated from the blood after dosing (plasma half-life 1-3 hours) and consecutive pulses of drug may be more beneficial than constant release for some treatments. The multiple-unit formulation disclosed comprises four identical hydrophilic matrix tablets placed in a gelatin capsule. Although the in vivo studies show two peaks in the plasma profile there is no well defined wash out period and the variation between the peak and trough plasma levels is small.

Conte et al., Drug Dev. Ind. Pharm, (1989) 15:2583-2596 and EP 0 274 734 (Pharmidea Srl) teach the use of a three layer tablet for delivery of ibuprofen in consecutive pulses. The three layer tablet is made up of a first layer containing the active ingredient, a barrier layer (the second layer) of semi-permeable material which is interposed between the first layer and a third layer containing an additional amount of active ingredient. The barrier layer and the third layer are housed in an impermeable casing. The first layer dissolves upon contact with a dissolving fluid while the third layer is only available after dissolution or rupture of the barrier layer. In such a tablet the first portion of active ingredient must be released instantly. This approach also requires the provision of a semi-permeable layer between the first and third layers in order to control the relative rates of delivery of the two portions of active ingredient. Additionally, rupture of the semi-permeable layer leads to uncontrolled dumping of the second portion of the active ingredient which may not be desirable.

U.S. Pat. No. 5,158,777 (E. R. Squibb & Sons Inc.) discloses a formulation comprising captopril within an enteric or delayed release coated pH stable core combined with additional captopril which is available for immediate release following administration. In order to form the pH stable core, chelating agents such as disodium edetate or surfactants such as polysorbate 80 are used either alone or in combination with a buffering agent. The compositions have an amount of captopril available for immediate release following oral administration and an additional amount of pH stabilized captopril available for release in the colon.

U.S. Pat. Nos. 4,728,512, 4,794,001 and 4,904,476 (American Home Products Corp.) relate to preparations providing three distinct releases. The preparation contains three groups of spheroids containing an active medicinal substance: the first group of spheroids is uncoated and rapidly disintegrates upon ingestion to release an initial dose of medicinal substance; the second group of spheroids is coated with a pH sensitive coat to provide a second dose; and the third group of spheroids is coated with a pH independent coat to provide to third dose. The preparation is designed to provide repeated release of medicinal substances which are extensively metabolized presystemically or have relatively short elimination half-lives.

U.S. Pat. No. 5,837,284 (Mehta et al) discloses a methylphenidate dosage form having immediate release and delayed release particles. The delayed release is provided by the use of ammonio methacrylate pH independent polymers combined with certain fillers.

Accordingly, it is an object of the present invention to provide a multiparticulate modified release composition comprising at least two populations of active ingredient-containing particles which, upon administration to a patient, exhibits a bimodal or multimodal release profile.

It is another object of the invention to provide a multiparticulate modified release composition comprising at least two populations of active ingredient-containing particles which, upon administration to a patient, exhibits a bimodal or multimodal release profile that results in a plasma profile within therapeutically effective pharmacokinetic parameters.

It is a further object of the invention to provide a multiparticulate modified release composition comprising at least two populations of active ingredient-containing particles which, upon administration to a patient, exhibits a pulsatile release profile.

It is yet another object of the invention to provide a multiparticulate modified release composition comprising at least two populations of active ingredient-containing particles which, upon administration to a patient, results in a pulsatile plasma profile.

It is still another object of the invention to provide a multiparticulate modified release composition comprising at least two populations of active ingredient-containing particles which, upon administration to a patient, produces a plasma profile substantially similar to the plasma profile produced by the administration of two or more IR dosage forms given sequentially.

It is yet a further object of the invention to provide a multiparticulate modified release composition comprising at least two populations of active ingredient-containing particles which, upon administration to a patient, substantially mimics the pharmacological and therapeutic effects produced by the administration of two or more IR dosage forms given sequentially.

It is still a further object of the invention to provide a multiparticulate modified release composition comprising at least two populations of active ingredient-containing particles in which the amount of the one or more active ingredients in the first population of particles is a minor portion of the amount of the one or more active ingredients in the composition, and the amount of the one or more active ingredients in the one or more additional population of particles is a major portion of the amount of the one or more active ingredients in the composition.

It is yet a further object of the invention to provide a solid oral dosage form comprising the multiparticulate modified release composition of the present invention.

Still other objects and advantages of the present invention will become readily apparent to those skilled in the art from the following detailed description, wherein the preferred embodiments of the invention are shown and described, simply by way of illustration of the best mode contemplated of carrying out the invention. As will be realized, the invention is capable of other and different embodiments, and its several details are capable of modifications in various obvious respects, all without departing from the invention.

### BRIEF DESCRIPTION OF THE INVENTION

The above objects are realized by a multiparticulate modified release composition having a first component comprising a first population of active ingredient-containing particles and at least a second component comprising a second population of active ingredient-containing particles. Each population of active ingredient-containing particles may comprise a single active ingredient or a combination of two or more active ingredients, and populations of particles comprising the composition may contain the same or different active ingredients. The active ingredient-containing particles of the at least second component are provided in a modified release (MR) form such as, for example, coated with a modified release coating or comprising or incorporated in a modified release matrix material. Upon oral administration to a patient, the composition releases the active ingredients in a bimodal or multimodal manner. As used herein, the term “active ingredient” includes a single active ingredient as well as combinations of two or more active ingredients.

The first component of the multiparticulate modified release composition may exhibit a variety of release profiles including profiles in which substantially all of the active ingredient contained in the first component is released rapidly upon administration of the dosage form, released rapidly but after a time delay (delayed release), or released slowly over time. In one embodiment, the active ingredient contained in the first component of the dosage form is released rapidly upon administration to a patient. As used herein, “released rapidly” includes release profiles in which at least about 80% of the active ingredient of a component of the dosage form is released within about an hour after administration, the term “delayed release” includes release profiles in which the active ingredient of a component of the dosage form is released (rapidly or slowly) after a time delay, and the terms “controlled release” and “extended release” include release profiles in which at least about 80% of the active ingredient contained in a component of the dosage form is released slowly.

The second component of the multiparticulate modified release composition may also exhibit a variety of release profiles including an immediate release profile, a delayed release profile or a controlled release profile. In one embodiment, the second component exhibits a delayed release profile in which the active ingredient of the component is released after a time delay. In another embodiment, the second component exhibits a controlled release profile in which the active ingredient of the component is released over a period of about 24 hours after administration.

In two-component embodiments in which the components exhibit different release profiles, the release profile of the active ingredients from the composition is bimodal. In embodiments in which the first component exhibits an immediate release profile and the second component exhibits a delayed release profile, there is a lag time between the release of active ingredient from the first component and the release of the active ingredient from the second component. The duration of the lag time may be varied by altering the amount and/or composition of the modified release coating or by altering the amount and/or composition of the modified release matrix material utilized to achieve the desired release profile.

In embodiments in which the first component exhibits an immediate release profile and the second component exhibits a controlled release profile, the active ingredients in the first and second components are released over different time periods. In one such embodiment, the active ingredient in the first component is released rapidly and the active ingredient in the second component is released within a period of about 12 hours after administration. In another such embodiment, the active ingredient in the first component is released rapidly and the active ingredient in the second component is released within a period of about 24 hours after administration. In yet another such embodiment, the active ingredient in the first component is released rapidly and the active ingredient in the second component is released over a period of about 12 hours after administration. In still another such embodiment, the active ingredient in the first component is released rapidly and the active ingredient in the second component is released over a period of about 24 hours after administration. In yet another such embodiment, the active ingredient in the first component is released rapidly and the active ingredient in the second component is released over a period of at least about 12 hours after administration. In still another such embodiment, the active ingredient in the first component is released rapidly and the active ingredient in the second component is released over a period of at least about 24 hours after administration.

The plasma profile produced by the administration of dosage forms of the present invention which comprise an immediate release component and at least one modified release component can be substantially similar to the plasma profile produced by the administration of two or more IR dosage forms given sequentially, or to the plasma profile produced by the administration of separate IR and MR dosage forms. Accordingly, the dosage forms of the present invention can be particularly useful for administering active ingredients for which patient tolerance is a potential or where the maintenance of pharmacokinetic parameters may be desired but is problematic.

In one embodiment of the present invention, the active ingredient is hydrocodone or pharmaceutically acceptable salts thereof, either alone or in combination with acetaminophen, and the composition, upon administration to a patient, releases the active ingredient in a bimodal manner. Such bimodal release results in a plasma profile in which pharmacokinetic values can be maintained within desired parameters.

The present invention also provides solid oral dosage forms made from the composition of the invention, and for methods for treating an animal, particularly a human, in need of treatment, comprising administering a dosage form comprising a therapeutically effective amount of the composition of the invention to provide bimodal or multimodal release of the active ingredient contained therein.

Advantages of the present invention include reducing the required dosing frequency while still maintaining the benefits derived from a bimodal or multimodal plasma profile. It is also advantageous in terms of patient compliance to have a formulation which may be administered at reduced frequency.

### DESCRIPTION OF THE DRAWINGS

FIG. 1 shows methylphenidate plasma profiles following oral administration of the following three formulations to human volunteers: A20 mg methylphenidate formulation having an immediate release component comprising particles containing a total of 10 mg methylphenidate (according to Table 1 (ii)) and a modified release component comprising particles containing a total of 10 mg methylphenidate (according to Table 2 (viii); IR particles coated to a 30% weight gain); B-20 mg methylphenidate formulation having an immediate release component comprising particles containing a total 10 mg methylphenidate (according to Table 1 (ii)) and a modified release component comprising particles containing a total of 10 mg methylphenidate (according to Table 2 (vii); IR particles coated to a 30% weight gain); and Control—two doses of 10 mg Ritalin® Hydrochloride (IR) tablets administered at times 0 and 4 hours (total of 20 mg methylphenidate administered).

FIG. 2 shows single dose simulations of 10 mg hydrocodone formulations of the present invention in which 20% of the hydrocodone is contained in the IR component.

FIG. 3 shows single dose simulations of 10 mg hydrocodone formulations of the present invention in which 20% of the hydrocodone is contained in the IR component.

FIG. 4 shows steady state simulations of 10 mg hydrocodone formulations of the present invention in which 20% of the hydrocodone is contained in the IR component.

FIG. 5 shows steady state simulations of 10 mg hydrocodone formulations of the present invention in which 20% of the hydrocodone is contained in the IR component.

FIG. 6 shows single dose simulations of 10 mg hydrocodone formulations of the present invention in which 50% of the hydrocodone is contained in the IR component.

FIG. 7 shows single dose simulations of 10 mg hydrocodone formulations of the present invention in which 50% of the hydrocodone is contained in the IR component.

FIG. 8 shows steady state simulations of 10 mg hydrocodone formulations of the present invention in which 50% of the hydrocodone is contained in the IR component.

FIG. 9 shows steady state simulations of 10 mg hydrocodone formulations of the present invention in which 50% of the hydrocodone is contained in the IR component.

FIG. 10 shows single dose simulations of 20-160 mg/day hydrocodone formulations of the present invention (Option 1) in which 20% of the hydrocodone is contained in the IR component.

FIG. 11 shows steady state simulations of 20-160 mg/day hydrocodone formulations of the present invention (Option 1) in which 20% of the hydrocodone is contained in the IR component.

FIG. 12 shows single dose simulations of 20-80 mg BID hydrocodone formulations of the present invention (Option 3) in which 20% of the hydrocodone is contained in the IR component.

FIG. 13 shows steady state simulations of 20-80 mg BID hydrocodone formulations of the present invention (Option 3) in which 20% of the hydrocodone is contained in the IR component.

FIG. 14 shows single dose simulations of 20-160 mg/day hydrocodone formulations of the present invention (Option 1) in which 50% of the hydrocodone is contained in the IR component.

FIG. 15 shows steady state simulations of 20-160 mg/day hydrocodone formulations of the present invention (Option 1) in which 50% of the hydrocodone is contained in the IR component.

FIG. 16 shows single dose simulations of 20-160 mg/day hydrocodone formulations of the present invention (Option 3) in which 50% of the hydrocodone is contained in the IR component.

FIG. 17 shows steady state simulations of 20-160 mg/day hydrocodone formulations of the present invention (Option 3) in which 50% of the hydrocodone is contained in the IR component.

### DETAILED DESCRIPTION OF THE INVENTION

The term “particulate” as used herein refers to a state of matter which is characterized by the presence of discrete particles, pellets, beads or granules irrespective of their size, shape or morphology. The term “multiparticulate” as used herein means a plurality of discrete or aggregated particles, pellets, beads, granules, or mixtures thereof, irrespective of their size, shape or morphology.

The term “modified release” as used herein includes a release which is not immediate and includes controlled release, extended release, sustained release and delayed release.

The term “time delay” as used herein refers to the period of time between the administration of a dosage form comprising the composition of the invention and the release of the active ingredient from a particular component thereof.

The term “lag time” as used herein refers to the time between the release of the active ingredient from one component of the composition and the release of the active ingredient from another component of the composition.

While exemplary embodiments of the invention will be described in detail with respect to compositions and dosage forms comprising either methylphenidate or hydrocodone as the active ingredient, the multiparticulate modified release compositions and dosage forms of the present invention are suitable for the delivery of any active ingredient or combination of active ingredients for which a bimodal or multimodal release results in a desired plasma profile.

The multiparticulate modified release composition and dosage forms made therefrom comprise at least two active ingredient-containing components. In one embodiment, the release of the active ingredient from the second and subsequent components, if any, is modified such that there is a lag time between the release of active ingredient from the first component and each subsequent component. The number of pulses in the release profile arising from such a composition in operation will depend on the number of active ingredient containing components in the composition. For example, a composition containing two active ingredient-containing components will give rise to two pulses in the release profile, and a composition containing three active ingredient-containing components will give rise to up to three pulses in the release profile. In another embodiment, the release of the active ingredients from subsequent components is modified such that the release of active ingredients from the first component and each subsequent component begins substantially upon administration but over different periods of time and/or at different rates.

Any active ingredient for which it is useful to combine the advantages of a bimodal or multimodal release profile in order to achieve their associated plasma profiles with a reduced frequency dosage regime may be used in practice of the present invention. One class of active ingredients that are useful in the practice of the invention includes active ingredients whose pharmacological and/or therapeutic effects benefit from having a wash-out period between plasma concentration peaks, such as those active ingredients susceptible to the development of patient tolerance. Another class of active ingredients that are useful in the practice of the invention includes active ingredients whose pharmacological and/or therapeutic effects benefit from maintaining particular pharmacokinetic values in a patient within desired parameters over the dosing period.

Exemplary active ingredients include but are not limited to drug compounds acting on the central nervous system such as psychostimulants and cerebral stimulants, for example methylphenidate; aldosterone inhibitors such as spironolactone, eplerenone and analogs thereof; alkaloids; alpha/beta-blockers such as labetalol, carvedilol and analogs thereof; analgesics such as acetaminophen, tramadol and opioids such as morphine, codeine, thebaine, heroin, oxycodone, hydrocodone, dihydrocodiene, hydromorphone, oxymorphone, buprenorphine, etorphine, naloxone, nicomorphine, methadone, pethidine, fentanyl, alfentanil, sufentanil, remifentanil, carfentanyl, pentazocine, phenazocine, butorphanol, levorphanol and analogs thereof; anesthetics such as lidocaine and bupivacaine and analogs thereof; anorectics such as benzphetamine, diethylproprion, mazindol, phendimetrazine, and phentermine; anti-adrenergic agents such as centrally and peripherally acting anti-adrenergic agents and analogs thereof; anti-allergic agents; anti-anginal agents such as nitroglycerine and analogs thereof; anti-arrythmic agents such as moricizine, ibutilide, quinidine, procainamide, disopyramide, lidocaine, tocainide, flecainide, mexiletine, propafenone, bretylium, amiodarone, adenosine, dofetilide and analogs thereof; anti-asthmatic agents such as salbutamol and analogs thereof; antibiotics such as aminosalicylic acid, amoxicillin, amoxicillin and potassium clavulanate, ampicillin, ampicillin and sulbactam, azithromycin, bacampicillin, carbenicillin, carbenicillin indanyl sodium, capreomycin, cefadroxil, cefazolin, cefcapene pivoxil, cephalexin, cephalothin, cephapirin, cephacelor, cefprozil, cephadrine, cefamandole, cefonicide, ceforanide, cefuroxime, cefixime, cefoperazone, cefotaxime, cefpodoxime, ceftaxidime, ceftibuten, ceftizoxime, ceftriaxone, cefepime, cefinetazole, cefotetan, cefoxitin, ciprofloxacine, clarithromycin, clindamycin, clofazimine, cloxacillin, cotriamoxazole, cycloserine, dicloxacillin, dirithromycin, erythromycin, ethambutol, ethionamide, fosfomycin, imipenem, isoniazide, levofloxacine, lomefloxacine, loracarbef, methicillin, methenamine, metronidazole, metoclopramide, meziocillin, nafcillin, nalidixic acid, nitrofurantoin, norfloxacin, novobiocin, ofloxacin, oxacillin, penicillin, pentamidine, piperacillin, piperacillin and tazobactam, sparfloxacin, sulphacytine, sulphamerazine, sulphamethazine, sulphamethixole, sulphasalazine, sulphisoxazole, sulphapyrizine, sulphadiazine, sulphmethoxazole, sulphapyridine, ticarcillin, ticarcillin and potassium clavulanate, trimethoprime, trimetrexate, troleanomycin, vancomycin, verapamil and analogs thereof; anti-cancer agents; anti-coagulant agents such as heparin, hirudin and analogs thereof; anti-convulsants such as carbamazepine, levetiracetam, topiramate and analogs thereof; anti-depressant agents such as amitriptyline, amoxapine, bupropion, citalopram, clomipramine, desipramine, doxepin, escitalopram, fluoxetine, fluvoxamine, imipramine, maprotiline, mirtazapine, nefazodone, nortriptyline, paroxetine, phenelzine, protriptyline, sertraline, tranylcypromine, trazodone, trimipramine, venlafaxine, and analogs thereof; anti-diabetic agents; anti-diarrheal agents such as loperamide and analogs thereof; anti-emetic agents such as scopolamine, ondansetron, domperidone, metoclopramide and analogs thereof; anti-epileptic agents; anti-fungal agents such as acylanilide and analogs thereof; antihistamines such as terfenadine and analogs thereof; anti-hypertensive agents; anti-inflammatory agents; anti-migraine agents such as sumatriptan, ergot alkaloids and analogs thereof; anti-neoplastics such as fluorouracil, bleomycin and analogs thereof; anti-parkinsonian agents; anti-psychotic agents such as acetophenazine, aripiprazole, chlorprothixene, droperidol, olanzapine, promazine, quetiapine, risperidone, sulpiride, triflupromazine, ziprasidone, and analogs thereof; anti-rheumatic agents such as fentiazac and analogs thereof; anti-thrombic agents; anti-tussive agents; anti-ulcer agents such as 5-asa, cimetidine, famotidine, lansoprazole, omeprazole, ranitidine and analogs thereof; anti-viral agents such as acyclovir, famciclovir, ganciclovir, zidovudine and analogs thereof; anxiolytic agents such as alprazolam, buspirone, clonazepam, clorazepate, chlordiazepoxide, diazepam, hydroxyzine, lorazepam, meprobamate, oxazepam, and analogs thereof; ARB blockers, such as irbesartan, candesartan, losartan, valsartan, telmisartan, eprosartan and analogs thereof; beta-blockers, such as acebutolol, atenolol, betaxolol, bisoprolol, esmolol, metoprolol, carteolol, nadolol, penbutolol, pindolol, propanolol, sotalol, timolol, labetalol and analogs thereof; blood lipid-lowering agents such statins such as simvastatin and analogs thereof; calcium channel blockers such as nifedipine, verapamil, diltiazem, nicardipine, nisoldipine, nimodipine, isradipine, bepridil, felodipine, amlodipine and analogs thereof; cardiovascular agents, anti-hypertensive agents and vasodilators such as benazepril, captopril, clonidine, enelapril, fosinopril, isosorbide dinitrate, isosorbide-5-mononitrate, hydralizine, lisinopril, moexipril, pentoxifylline, perindopril, prazosine, quinapril, quinidine, ramipril, trandolapril, nitrates, peripheral vasodilators and analogs thereof; chelating agents such as deferoxamine and analogs thereof; chemotherapy agents such as vincristine and analogs thereof; contraceptives; diuretic agents such as loop diuretics, acetazolamide, amiloride, bendroflumethiazide, bumetanide, chlorthalidone, chlorothiazide, dichlorphenamide, ethacrynic acid, furoseamide, hydrochlorothiazide, hydroflumethiazide, indapamide, mannitol, methazolamide, methyclothiazide, metolazone, naturetin, polythiazide, spironolactone, triameterene, triamterene, trichlormethiazide, triamterene, torsemide, and analogs thereof; fertility promoters; hypnotic agents such as amobarbital, butabarbital, chloral hydrate, estazolam, flurazepam, mephobarbital, paraldehyde, pentobarbital, phenobarbital, quazepam, secobarbital, temazepam, triazolam, zaleplon, zolpidem and analogs thereof; inducers and inhibitors of uterine labor; inotropic agents such as digoxin and analogs thereof; narcotic antagonists; NSAIDs such as celecoxib, etoricoxib, rofecoxib, valdecoxib, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, tiaprofenic acid, salicylates such as acetylsalicylic acid, choline magnesium salicylate, choline salicylate, magnesium salicylate, and sodium salicylate, and analogs thereof; neuroleptic agents; synthetic and naturally occurring peptides, proteins or hormones such as desmopressin, vasopressin, insulin, calcitonin, calcitonin gene regulating protein, atrial natriuretic protein, colony stimulating factor, betaseron, erythropoietin (EPO), interferons such as α, β or γ interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2 and analogs thereof; prostaglandins and analogs thereof; sedatives such as benzodiazepines, phenothiozines and analogs thereof; and vasoprotective agents.

It will be understood that suitable active ingredients also include all pharmaceutically acceptable salts, acids, esters, complexes or other derivatives of the active ingredients recited above, and may be present either in the form of one enantiomer or as a mixture, racemic or otherwise, of enantiomers.

The active ingredient in each component may be the same or different. In one embodiment, the first component contains a first active ingredient and the second component comprises a second active ingredient. In another embodiment, two or more active ingredients may be incorporated into one or more components. Further, an active ingredient present in one component of the composition may be accompanied by, for example, an enhancer compound or a sensitizer compound in another component of the composition, in order to modify the bioavailability or therapeutic effect of the active ingredient.

As used herein, the term “enhancer” refers to a compound which is capable of enhancing the absorption and/or bioavailability of an active ingredient by promoting net transport across the GIT in an animal, such as a human. Enhancers include but are not limited to medium chain fatty acids and salts, esters, ethers and derivatives thereof, including glycerides and triglycerides; non-ionic surfactants such as those that can be prepared by reacting ethylene oxide with a fatty acid, a fatty alcohol, an alkylphenol or a sorbitan or glycerol fatty acid ester; cytochrome P450 inhibitors, P-glycoprotein inhibitors and the like; and mixtures thereof.

The amount of the active ingredient contained in the composition and in dosage forms made therefrom may be allocated evenly or unevenly across the different particle populations comprising the components of the composition and contained in the dosage forms made therefrom. In one embodiment, the active ingredient contained in the particles of the first component comprises a minor portion of the total amount of active ingredient in the composition or dosage form, and the amount of the active ingredient in the other components comprises a major portion of the total amount of active ingredient in the composition or dosage form. In one such embodiment comprising two components, about 20% of the total amount of the active ingredient is contained in the particles of the first component, and about 80% of the total amount of the active ingredient is contained in the particles of the second component.

The active ingredient is preferably present in the composition and in dosage forms made therefrom in an amount of from about 0.1 to about 1000 mg, preferably in the amount of from about 1 to about 160 mg, and more preferably from about 5 to about 80 mg. Depending at least in part on the particular active ingredients that are included in the composition and dosage forms, the active ingredient is present in an amount of from about 5 to about 80 mg, about 5 to about 60 mg, about 5 to about 40 mg, about 5 to about 20 mg, about 5 to about 10 mg, about 10 to about 80 mg, about 10 to about 60 mg, about 10 to about 40 mg, about 10 to about 20 mg, about 20 to about 80 mg, about 20 to about 60 mg, about 20 to about 40 mg, about 40 to about 80 mg, about 40 to about 60 mg, and about 60 to about 80 mg.

When the active ingredient is methylphenidate, it is preferably present in the composition and in dosage forms made therefrom in an amount of from about 0.5 to about 60 mg; more preferably the active ingredient is present in the first component in an amount of from about 2.5 to about 30 mg. When the active ingredient is hydrocodone, it is preferably present in the composition and in dosage forms made therefrom in an amount of from about 5 to about 160 mg; more preferably the active ingredient is present in the first component in an amount of from about 10 to about 80 mg.

The profile for the release of the active ingredient from each component of the composition may be varied by modifying the composition of each component, including modifying any of the excipients or coatings which may be present. In particular the release of the active may be controlled by the choice and amount of the modified release coating applied to the particles where such a coating is present. If more than one modified release component is present, the modified release coating for each of these components may be the same or different. Similarly, when the modified release is accomplished by means of a modified release matrix material, release of the active ingredient may be controlled by the choice and amount of modified release matrix material utilized.

In one embodiment, the first component may be an immediate release component wherein the active ingredient contained therein is released substantially immediately upon administration. In another embodiment, the first component may be a delayed release component in which the active ingredient is released substantially immediately after a time delay. In either of such embodiments, the second component may be a modified release component in which the active ingredient is released over a period of time or substantially immediately after a time delay.

As will be appreciated by those skilled in the art, the exact nature of the plasma profile will be influenced by the combination of all of the factors described above. Thus by variation of the composition of each component thereof, including the amount and nature of the active ingredient and the modified release coating or modified matrix material, if any, numerous plasma profiles may result therefrom upon administration to a patient. Depending on the release profile of each component, the plasma profile resulting therefrom may be bimodal or multimodal, and may define well separated and clearly defined peaks associated with each with each component (e.g. when the lag time between immediate release and delayed release components is long) or superimposed peaks associated with each component (e.g. in when the lag time is short). For example, administration of a multiparticulate modified release composition having an immediate release component and a single modified release component can result in a plasma profile in which the immediate release component of the composition gives rise to a first peak in the plasma profile and the modified release component gives rise to a second peak in the plasma profile. Embodiments of the invention comprising more than one modified release component may give rise to further peaks in the plasma profile. Alternatively, administration of a multiparticulate modified release composition having an immediate release component and one or more modified release components can result in a bimodal or multimodal release profile but a plasma profile having a single peak or peaks fewer in number than the number of components contained in the composition.

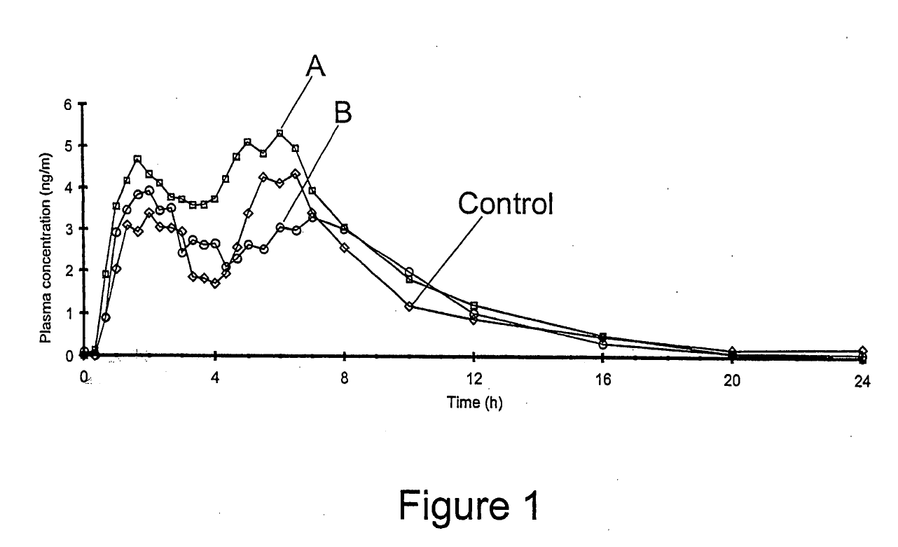

The plasma profile produced from the administration of a single dosage unit of the present invention is advantageous when it is desirable to deliver two or more portions of active ingredient without the need for administration of two or more dosage units. Additionally, in the case of some disorders it is particularly useful to have such a bimodal plasma profile. For example, a typical methylphenidate treatment regime consists of administration of two doses of an immediate release dosage formulation given four hours apart. This type of regime has been found to be therapeutically effective and is widely used. The plasma profile produced by such an administration regime is illustrated by the “Control” curve in FIG. 1. As previously mentioned, the development of patient tolerance is an adverse effect sometimes associated with methylphenidate treatments. It is believed that the trough in the plasma profile between the two peak plasma concentrations is advantageous in reducing the development of patient tolerance by providing a period of wash out of the active ingredient. Drug delivery systems which provide zero order or pseudo zero order delivery of methylphenidate do not facilitate this wash out process.

In embodiments which include drug compounds used for pain management, such as for example hydrocodone, the compositions and dosage forms of the present invention may provide continuous analgesia for up to 24 hours by providing minimum peak to trough fluctuations in plasma levels and reduce or eliminate side effects associated with such drug compounds.

Any coating material which modifies the release of the active ingredient in the desired manner may be used in the practice of the present invention. In particular, coating materials suitable for use in the practice of the invention include but are not limited to polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark Eudragit® RS and RL, poly acrylic acid and poly acrylate and methacrylate copolymers such as those sold under the trademark Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, poly vinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, aminoacryl-methacrylate copolymer (Eudragit® RS-PM, Rohm & Haas), pullulan, collagen, casein, agar, gum arabic, sodium carboxymethyl cellulose, (swellable hydrophilic polymers) poly(hydroxyalkyl methacrylate) (m. wt. ˜5 k-5,000 k), polyvinylpyrrolidone (m. wt. ˜10 k-360 k), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (m. wt. ˜30 k-300 k), polysaccharides such as agar, acacia, karaya, tragacanth, algins and guar, polyacrylamides, Polyox® polyethylene oxides (m. wt. ˜100 k-5,000 k), AquaKeep® acrylate polymers, diesters of polyglucan, crosslinked polyvinyl alcohol and poly N-vinyl-2-pyrrolidone, sodium starch glycolate (e.g. Explotab®; Edward Mandell C. Ltd.); hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox®, Union Carbide), methyl ethyl cellulose, ethylhydroxy ethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®, Rohm and Haas), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof.

Excipients such as plasticisers, lubricants, solvents and the like may be added to the coating. Suitable plasticisers include for example acetylated monoglycerides; butyl phthalyl butyl glycolate; dibutyl tartrate; diethyl phthalate; dimethyl phthalate; ethyl phthalyl ethyl glycolate; glycerin; propylene glycol; triacetin; citrate; tripropioin; diacetin; dibutyl phthalate; acetyl monoglyceride; polyethylene glycols; castor oil; triethyl citrate; polyhydric alcohols, glycerol, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidized tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate.

When the modified release component comprises a modified release matrix material, any suitable modified release matrix material or suitable combination of modified release matrix materials may be used. Such materials are known to those skilled in the art. The term “modified release matrix material” as used herein includes hydrophilic polymers, hydrophobic polymers and mixtures thereof which are capable of modifying the release of an active ingredient dispersed therein in vitro or in vivo. Modified release matrix materials suitable for the practice of the present invention include but are not limited to microcrystalline cellulose, sodium carboxymethylcellulose, hydroxyalkylcelluloses such as hydroxypropylmethyl-cellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

A multiparticulate modified release composition according to the present invention may be incorporated into any suitable dosage form which facilitates release of the active ingredient in a bimodal or multimodal manner. Typically, the dosage form may be a blend of the different populations of active ingredient containing particles which make up the immediate release and the modified release components, the blend being filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multilayer tablet. In such dosage forms, the first component of the multiparticulate modified release composition may be compressed into one layer with the second component being subsequently added as a second layer of the multilayer tablet. The populations of active ingredient containing particles comprising the composition of the invention may further be included in rapidly dissolving dosage forms such as an effervescent dosage form or a fast-melt dosage form.

In one embodiment, the composition of the invention and the dosage forms made therefrom release the active ingredient such that substantially all active ingredient contained in the first component is released prior to release of active ingredient from the second component. For example, when the first component comprises an IR component, release of the active ingredient from the second component may be delayed until substantially all the active ingredient in the IR component has been released. Release of the active ingredient from the second component may be delayed as detailed above by the use of a modified release coating and/or a modified release matrix material.

When it is desirable to minimize patient tolerance by providing a dosage regime which facilitates wash-out of a first dose of active ingredient from a patient's system, release of the active ingredient from the second component is delayed until substantially all of the active ingredient contained in the first component has been released, and further delayed until at least a portion of the active ingredient released from the first component has been cleared from the patient's system. In one embodiment, release of the active ingredient from the second component of the composition is substantially, if not completely, delayed for a period of at least about two hours after administration of the composition. In one such embodiment in which the active ingredient is methylphenidate, release of the active ingredient from the second component of the composition is substantially, if not completely, delayed for a period of at least about four hours, preferably about four hours, after administration of the composition.

In another embodiment, the composition of the invention and the dosage forms made therefrom release the active ingredient such that the active ingredient contained in the first component is released during the release of active ingredient from the second component. In one such embodiment in which the active ingredient is hydrocodone, release of the active ingredient from the second component of the composition occurs during and beyond the release of the active ingredient from the first component.

In the following Examples all percentages are weight by weight unless otherwise stated. The term “purified water” as used throughout the Examples refers to water that has been purified by passing it through a water filtration system.

### EXAMPLE 1

Multiparticulate Modified Release Composition Containing Methylphenidate

A multiparticulate modified release composition according to the present invention comprising an immediate release component and a modified release component and containing methylphenidate as the active ingredient is prepared as follows.

(a) Immediate Release Component.

A solution of methylphenidate HCl (50:50 racemic mixture) is prepared according to any of the formulations given in Table 1. The methylphenidate solution is then coated onto nonpareil seeds to a level of approximately 16.9% solids weight gain using, for example, a Glatt GPCG3 (Glatt, Protech Ltd., Leicester, UK) fluid bed coating apparatus to form the IR particles of the immediate release component.

TABLE 1
Immediate release component solutions

Amount,
% (w/w)

| Ingredient | (i) | (ii) |
|---|---|---|
| Methylphenidate HCl | 13.0 | 13.0 |
| Polyethylene Glycol 6000 | 0.5 | 0.5 |
| Polyvinylpyrrolidone | 3.5 | |
| Purified Water | 83.5 | 86.5 |

(b) Modified Release Component.

Methylphenidate containing delayed release particles are prepared by coating immediate release particles prepared according to Example 1(a) above with a modified release coating solution as detailed in Table 2. The immediate release particles are coated to varying levels up to approximately to 30% weight gain using, for example, a fluid bed apparatus.

TABLE 2
Modified release component coating solutions

| | Amount, % (w/w) | | | | | | | |
|---|---|---|---|---|---|---|---|---|
| Ingredient | (i) | (ii) | (iii) | (iv) | (v) | (vi) | (vii) | (viii) |
| Eudragit ® RS | 49.7 | 42.0 | 47.1 | 53.2 | 40.6 | — | — | 25.0 |
| 12.5 | | | | | | | | |
| Eudragit ® S | — | — | — | — | | 54.35 | 46.5 | — |
| 12.5 | | | | | | | | |
| Eudragit ® L | — | — | — | — | — | — | — | 25.0 |
| 12.5 | | | | | | | | |
| Polyvinyl- | — | — | — | 0.35 | 0.3 | — | — | — |
| pyrrolidone | | | | | | | | |
| Diethyl- | 0.5 | 0.5 | 0.6 | 1.35 | 0.6 | 1.3 | 1.1 | — |
| phthalate | | | | | | | | |
| Triethylcitrate | — | — | — | — | — | — | — | 1.25 |
| Isopropyl | 39.8 | 33.1 | 37.2 | 45.1 | 33.8 | 44.35 | 49.6 | 46.5 |
| alcohol | | | | | | | | |
| Acetone | 10.0 | 8.3 | 9.3 | — | 8.4 | — | — | — |
| Talc¹ | — | 16.0 | 5.9 | — | 16.3 | — | 2.8 | 2.25 |

¹Talc is simultaneously applied during coating for formulations in column (i), (iv) and (vi).

(c) Dissolution Testing

pH independent coated components ((i) to (v) Table 2) are tested in vitro in USP Type 1 apparatus (100 rpm) according to the following protocol: the sample is placed in 0.01 N HCl (900 ml), pH 2.0, 37° C. for all of the sampling time points.

pH dependent coated components ((vi) to (viii) Table 2) are tested in USP Type 1 apparatus (100 rpm) according to a modified version of the United States Pharmacopoeia method for enteric protection (U.S. Pat. No. 23, 1995, p. 1795): the sample is placed for 2 hours in 0.01 N HCl and then transferred to phosphate buffer pH 6.8 for the remainder of the sampling time points.

IR components were formulated using three different sizes of non-pareil seeds having diameter dimensions of 0.5-0.6, 0.6-0.71 and 0.71-0.85 mm, respectively. The IR particles formed by coating 0.5-0.6, 0.6-0.71 and 0.71-0.85 mm nonpareil seeds were found to release 100% of the active ingredient within 20 minutes in aqueous media.

Dissolution data for the modified release components prepared according to Example 1(b) above are shown in Tables 3 (a) to 3 (c). This data shows that release characteristics of the modified release component can be varied by changing the composition and thickness of the coating applied.

TABLE 3 (a)
Dissolution data for modified release components formulated with
coating solutions given in Table 2

| | Coating formulation | | | | | | | |
|---|---|---|---|---|---|---|---|---|
| | (i) | (i) | (i) | (ii) | (ii) | (ii) | (iii) | (iii) |
| | Coating level (% weight gain) | | | | | | | |
| | 4% | 6% | 10% | 4% | 6% | 8% | 4% | 6% |
| Time (hr) | % Active ingredient released | | | | | | | |
| 1 | 0 | 0 | 0 | 8.5 | 1.3 | 1.4 | 6.1 | 3.0 |
| 2 | 17.0 | 3.3 | 0 | 36.9 | 7.1 | 3.7 | 21.3 | 8.2 |
| 4 | 51.5 | 22.1 | 0 | 80.0 | 40.3 | 15.1 | 62.3 | 26.3 |
| 6 | 75.8 | 46.5 | 0 | 92.8 | 72.4 | 31.2 | 82.1 | 52.6 |
| 8 | 86.0 | 65.5 | 10.2 | 97.5 | 83.0 | 47.5 | 91.3 | 73.0 |
| 10 | 91.3 | 76.5 | 17.3 | — | — | — | 97.7 | 86.5 |

(the notation “—” indicates no measurement taken)

TABLE 3 (b)
Dissolution data for modified release components formulated
with coating solutions given in Table 2

| | Coating formulation | | | | |
|---|---|---|---|---|---|
| | (iv) | (iv) | (iv) | (v) | (v) |
| | Coating level (% weight gain) | | | | |
| | 10% | 15% | 20% | 10% | 12.5% |
| Time (hr) | % Active ingredient released | | | | |
| 1 | 3.5 | 0.9 | 1.1 | 1.3 | 1.0 |
| 2 | 13.4 | 5.4 | 2.9 | 6.1 | 2.9 |
| 4 | 47.1 | 22.5 | 13.8 | 42.4 | 21.2 |
| 6 | 80.0 | 52.0 | 36.9 | 77.5 | 54.4 |
| 8 | 94.8 | 70.3 | 61.0 | 92.4 | 79.7 |
| 10 | 103 | 81.5 | 76.1 | — | — |

(the notation “—” indicates no measurement taken)

TABLE 3 (c)
Dissolution data for modified release components formulated
with coating solutions given in Table 2

| | Coating formulation | | | | | | | |
|---|---|---|---|---|---|---|---|---|
| | (vi) | (vi) | (vi) | (vi)* | (vii) | (vii) | (viii) | (viii) |
| | Coating level (% weight gain) | | | | | | | |
| | 5% | 10% | 15% | 15% | 15% | 20% | 20% | 30% |
| Time (hr) | % Active ingredient released | | | | | | | |
| 1 | 33.2 | 0.4 | 0 | 0 | 3.9 | 0.6 | 3.8 | 2.1 |
| 2 | 80.6 | 9.8 | 0 | 0.5 | 52.0 | 12.4 | 7.4 | 3.1 |
| 4 | 92.2 | 43.5 | 10.1 | 44.0 | 85.0 | 61.6 | 43.7 | 8.9 |
| 6 | 93.9 | 61.6 | 29.9 | 80.2 | 89.9 | 75.3 | 72.4 | 36.9 |
| 8 | 94.3 | 67.5 | 48.4 | 69.0 | 91.4 | 79.6 | 79.2 | 63.9 |
| 10 | 94.4 | — | 60.0 | — | — | — | 79.5 | 73.4 |

(the notation “—” indicates no measurement taken; “*” indicates pH of phosphate buffer was 7.4 instead of 6.8)

(d) Encapsulation of Immediate and Delayed Release Particles.

The immediate and delayed release particles prepared according to Example 1(a) and (b) above are encapsulated in size 2 hard gelatin capsules to an overall 20 mg dosage strength using, for example, a Bosch GKF 4000S encapsulation apparatus. The overall dosage strength of 20 mg methylphenidate was made up of 10 mg from the immediate release component and 10 mg from the modified release component.

Table 4 shows the dissolution profiles for two multiparticulate modified release compositions prepared using the immediate release coating solution given in Table 1 (ii) and the modified release coating solutions given in Table 2 (vii) and (viii). These results indicate that approximately 50% of the methylphenidate HCl active ingredient was released within the first half hour with release from the modified release component being delayed for about four hours.

TABLE 4
Dissolution data for compositions containing an
IR component and a modified release component

MR coating formulation

| | |
|---|---|
| (vii) | (viii) |

Coating level
(% weight increase)

| | |
|---|---|
| 30% | 30% |

| | |
|---|---|
| Time (hr) | % Active ingredient released |

| | | |
|---|---|---|
| 0 | 0 | 0 |
| 0.5 | 49.7 | 50.2 |
| 1 | 49.7 | 50.5 |
| 2 | 49.8 | 51.1 |
| 4 | 56.1 | 54.1 |
| 6 | 65.2 | 68.0 |
| 8 | 72.2 | 81.8 |
| 10 | 76.6 | 87.0 |

The dissolution profiles shown in Table 4 indicate that the compositions containing the pH dependent coated components release the methylphenidate active ingredient in a pulsed manner. A first pulse occurs before 1 hour followed by a plateau region where the release of further amounts of the active ingredient is suppressed. The plateau region is in turn followed by a second pulse of active ingredient release as indicated by the increase in drug concentration from 4 hours onward.

### EXAMPLE 2

### Multiparticulate Modified Release Composition Containing Methylphenidate

Multiparticulate modified release methylphenidate compositions according to the present invention having an immediate release component and a modified release component having a modified release matrix material are prepared according to the formulations shown in Table 5 (a) and (b).

TABLE 5 (a)
100 mg of IR component is encapsulated with 100 mg of modified-
release (MR) component to give a 20 mg dosage strength product

| | % | | % |
|---|---|---|---|
| IR component | (w/w) | MR component | (w/w) |
| Methylphenidate HCl | 10 | Methylphenidate HCl | 10 |
| Microcrystalline cellulose | 40 | Microcrystalline cellulose | 40 |
| Lactose | 45 | Eudragit .RTM. RS | 45 |
| Povidone | 5 | Povidone | 5 |

TABLE 5 (1)
50 mg of IR component is encapsulated with 50 mg of modified-
release (MR) component to give a 20 mg dosage strength product

| | % | | % |
|---|---|---|---|
| IR component | (w/w) | MR component | (w/w) |
| Methylphenidate HCl | 20 | Methylphenidate HCl | 20 |
| Microcrystalline cellulose | 50 | Microcrystalline cellulose | 50 |
| Lactose | 28 | Eudragit ® RS | 28 |
| Povidone | 2 | Povidone | 2 |

(e) In Vivo Release

In a human cross-over basted, fasted healthy volunteers were dosed with 20 mg methylphenidate HCl compositions according to the present invention to compare the bioavailability of methylphenidate HCl in these compositions relative to Ritalin® (Novartis; 10 mg dosed twice at a four hour interval). Pharmacokinetic assessment was based on the plasma levels of methylphenidate measured by blood sampling at regular intervals up to 48 hours after administration. Blood samples were also taken for pre- and post-study screening.

Referring now to FIG. 1, the plasma profiles labeled “A” (modified component comprises IR particles coated with coating Table 2 (viii) at 30%) and “B” (modified component comprises IR particles coated with coating Table 2 (vii) at 30%) correspond to the plasma concentrations of methylphenidate observed in human volunteers after oral administration of the multiparticulate modified release compositions prepared according to Example 1. In both cases the plasma profile is qualitatively similar to the control, typical of prior art treatments (labeled “Control” in FIG. 1), which consists of two doses of Ritalin® IR given sequentially, four hours apart.

For the multiparticulate modified release composition according to the present invention prepared according to Example 1 above, the first peak in the plasma profile associated with the immediate release component is similar in terms of cₘₐₓ and peak width to the peak associated with the first dose of Ritalin® in the control profile. Profile A shows that the trough characteristic of the conventional twice daily administration (as exemplified by the control profile) is mimicked by the composition prepared according to the invention. Profile B also shows a significant fall off after the initial peak in plasma concentration. For both multiparticulate modified release compositions, the effect of the modified release component is to increase plasma concentrations four hours after administration resulting in a second peak level. This observed effect again mimics the control.

From FIG. 1 it is clear that certain of the multiparticulate modified release compositions prepared according to the present invention mimic a typical twice daily treatment (represented by the control) in terms of the plasma profile achieved upon administration. This in vivo release of methylphenidate from compositions according to the invention was achieved without any loss in bioavailability compared to Ritalin® dosed twice daily.

In a separate study, 34 children with ADHD were dosed with 20 mg methylphenidate HCl compositions according to the present invention. A simulated classroom design was used to compare formulations “A” and “B” (corresponding to the “A” and “B,” formulations described above) with placebo. Pharmacodynamic assessments were conducted over a 9 hour time period which measured both attention and deportment as measured on the SKAMP scale and functional outcome as measured by the number of math problems attempted and the number of correct answers. Each formulation demonstrated a statistical difference from placebo on all efficacy measurements. The individual efficacy evaluations showed that the “A” and “B” formulations proved to be similar with regard to deportment. With regard to attention and functional outcome, the children on the “A” formulation appeared to focus more on the tasks at hand and attempted more math problems more quickly between 4 and 6 hours than the children taking the “B” formulation.

### EXAMPLE 3

### Multiparticulate Modified Release Composition Containing Hydrocodone Bitartrate

Multiparticulate modified release hydrocodone compositions according to the present invention having an immediate release component and a modified release component having a modified release coating are prepared according to the formulations shown in Tables 6 and 7.

TABLE 6
Immediate Release Component Hydrocodone Solutions

| | Amount, % (w/w) | | | | | |
|---|---|---|---|---|---|---|
| Ingredient | (i) | (ii) | (iii) | (iv) | (v) | (vi) |
| Hydrocodone Bitartrate | 6.0 | 6.0 | 6.0 | 6.0 | 6.0 | 6.0 |
| HPMC 2910 | 1.0 | 2.0 | 2.0 | — | — | 1.5 |
| Polyethylene Glycol 6000 | — | — | — | 0.5 | — | — |
| Povidone K30 | — | — | | — | 5.0 | — |
| Fumaric Acid | — | 6.0 | — | — | — | — |
| Citric Acid | — | — | 6.0 | — | — | — |
| Silicon Dioxide | 1.5 | 1.0 | 1.0 | — | — | 2.0 |
| Talc | 1.5 | — | — | — | — | — |
| Purified Water | 90.0 | 85.0 | 85.0 | 93.5 | 89.0 | 90.5 |

TABLE 7
Modified Release Component Hydrocodone Solutions

| | Amount, % (w/w) | | | | | | |
|---|---|---|---|---|---|---|---|
| Ingredient | (i) | (ii) | (iii) | (iv) | (v) | (vi) | (vii) |
| Eudragit RS 100 | 4.1 | 4.9 | 5.5 | 4.4 | — | 5.5 | 7.5 |
| Eudragit RL 100 | — | 0.5 | — | 1.1 | — | — | — |
| Eudragit L 100 | 1.4 | — | — | — | — | — | — |
| Ethocel | — | — | — | — | 3.0 | — | — |
| Triethyl Citrate | 1.5 | 1.6 | — | 1.1 | — | — | 1.5 |
| Dibutyl Sebacate | — | — | — | — | 0.6 | 1.0 | — |
| Silicon Dioxide | 1.0 | 1.0 | 1.0 | — | 2.0 | 1.0 | — |
| Talc | 2.5 | 2.5 | 1.0 | 2.8 | — | 1.0 | 2.5 |
| Acetone | 34.0 | 34.0 | 15.0 | 35.6 | — | 14.0 | 33.5 |
| Isopropyl Alcohol | 50.0 | 50. | 72.5 | 50. | 94.4 | 72.5 | 50.0 |
| Purified Water | 5.5 | 5.5 | 5.0 | 5.0 | — | 5.0 | 5.0 |

In these exemplary hydrocodone formulations, the sugar spheres (30/35 mesh) are provided as inert cores that act as a carrier for the active ingredient and other excipients present in the formulation. The quality and size selected reflect the requirement to produce multiparticulates with a mean diameter in the size range 0.5-0.6 mm to facilitate the subsequent coating and encapsulation process. Hydroxypropylmethylcellulose (2910) (Methocal E6 Premium LV) is used to prepare the immediate-release coating solution that is coated onto the sugar spheres to produce the IR beads and acts as a binding agent. Silicon Dioxide (Syloid 244FP) is an anti-adherent that is used in the preparation of the IR coating solution (Table 6) and the modified release coating suspension (Table 7). Ammonio methyacrylate copolymer Type B (Eudragit RS 100) is a rate-controlling polymer that imparts the controlled release properties to the formulation and exhibits pH independent release properties. Talc (Altalc 200) is used as an anti-adherent in the modified-release coating process to manufacture the modified release beads. Acetone and isopropyl alcohol are the two solvents in which the rate-controlling polymer is dissolved to produce the coating suspension that is applied to the IR beads to form the modified release beads. The resultant coating suspension is applied to the IR beads to form the modified release beads. Modified release beads are dried in an oven for 10-20 hours at 40-500 C/30-60% RH to remove residual solvents and to obtain a moisture content of about 3-6%. Suitable processing procedures are further detailed in U.S. Pat. No. 6,066,339 which is incorporated herein by reference in its entirety.

Table 8 shows the dissolution profiles for two multiparticulate modified release formulations prepared in accordance with Tables 6 and 7. These results indicate that about 20 % of the hydrocodone was released in the first hour and about 80% of the hydrocodone was released over a period of about 11 hours.

TABLE 8
Dissolution Data for Compositions Containing an
IR Component and a Modified Release Component

Formulation

| Time (hr) | Fumaric Acid | Non-Fumaric Acid |
|---|---|---|
| 0 | 0 | 0 |
| 1 | 22 | 26 |
| 2 | 33 | 31 |
| 4 | 54 | 54 |
| 6 | 68 | 64 |
| 8 | 77 | 73 |
| 12 | 93 | 86 |

In Vivo Study

A randomized, single-dose, parallel-group, placebo-controlled, active-comparator study was performed to evaluate the safety, efficacy, and PK of hydrocodone formulations in subjects immediately following bunionectomy study. The study treatments were 10, 20, 30, 40 mg of hydrocodone bitartarate, matching active comparator (10 mg hydrocodone/APAP) or matching placebo. During the 24-hour confinement periods, blood was collected at baseline and at up to 17 additional time points, from 115 subjects (approx. 17 to 21 subjects per group), to determine the concentrations in plasma of hydrocodone. The following PK parameters were calculated and are presented in Tables 9-11.

TABLE 9

| | | HC ER 10 mg | HC ER 20 mg | HC ER 30 mg | HC ER 40 mg | HC/APAP | Placebo |
|---|---|---|---|---|---|---|---|
| Parameter | Statistics | N = 21 | N = 19 | N = 19 | N = 17 | N = 18 | N = 21 |
| Cmax (ng/mL) | n | 21 | 19 | 19 | 17 | 18 | 21 |
| | Mean | 8.9 | 17.9 | 31.7 | 37.5 | 19.5 | 0.1 |
| | Std. Dev. | 2.11 | 5.65 | 8.50 | 8.32 | 8.69 | 0.17 |
| | Median | 9.1 | 16.3 | 30.1 | 34.1 | 20.2 | 0.0 |
| | Min/Max | 5/15 | 10/27 | 18/46 | 28/62 | 9/45 | 0/1 |
| Tmax (hr) | n | 21 | 19 | 19 | 17 | 18 | 3 |
| | Mean | 6.3 | 6.0 | 6.3 | 5.1 | 2.7 | 8.2 |
| | Std. Dev. | 1.46 | 1.80 | 1.88 | 1.52 | 1.65 | 13.70 |
| | Median | 6.1 | 5.2 | 6.1 | 6.0 | 2.1 | 0.6 |
| | Min/Max | 4/9 | 4/12 | 4/10 | 4/10 | 1/7 | 0/24 |
| kel (1/hr) | n | 21 | 19 | 19 | 17 | 18 | NC (a) |
| | Mean | 0.090 | 0.095 | 0.086 | 0.079 | 0.138 | NC |
| | Std. Dev. | 0.0276 | 0.0289 | 0.0229 | 0.0211 | 0.0297 | NC |
| | Median | 0.092 | 0.089 | 0.083 | 0.079 | 0.147 | NC |
| | Min/Max | 0.02/0.13 | 0.05/0.16 | 0.05/0.13 | 0.05/0.13 | 0.06/0.18 | NC |

(a) NC = Not Calculated

TABLE 10

| | | HC ER 10 mg | HC ER 20 mg | HC ER 30 mg | HC ER 40 mg | HC/APAP | Placebo |
|---|---|---|---|---|---|---|---|
| Parameter | Statistics | N = 21 | N = 19 | N = 19 | N = 17 | N = 18 | N = 21 |
| t½ (hr) | n | 21 | 19 | 19 | 17 | 18 | NC |
| | Mean | 9.5 | 7.9 | 8.6 | 9.4 | 5.3 | NC |
| | Std. Dev. | 8.25 | 2.44 | 2.32 | 2.40 | 1.54 | NC |
| | Median | 7.6 | 7.8 | 8.4 | 8.8 | 4.7 | NC |
| | Min/Max | 5/45 | 4/15 | 5/13 | 5/14 | 4/11 | NC |
| AUClast (ng · hr/mL) | n | 21 | 19 | 19 | 17 | 18 | 21 |
| | Mean | 109.0 | 212.9 | 392.5 | 464.6 | 131.2 | 0.1 |
| | Std. Dev. | 27.25 | 73.19 | 117.74 | 124.01 | 36.80 | 0.19 |
| | Median | 104.2 | 196.2 | 367.0 | 471.0 | 129.9 | 0.0 |
| | Min/Max | 73/179 | 130/377 | 177/671 | 321/712 | 80/182 | 0/1 |
| AUCinf (ng · hr/mL) | n | 21 | 19 | 19 | 17 | 18 | NC |
| | Mean | 136.9 | 255.6 | 480.7 | 696.2 | 137.6 | NC |
| | Std. Dev. | 39.48 | 88.66 | 138.70 | 172.73 | 39.99 | NC |
| | Median | 128.1 | 252.7 | 459.5 | 578.0 | 135.4 | NC |
| | Min/Max | 80/217 | 151/468 | 226/756 | 375/992 | 83/189 | NC |

(a) NC = Not Calculated

TABLE 11

| | | HC ER 10 mg | HC ER 20 mg | HC ER 30 mg | HC ER 40 mg | HC/APAP | Placebo |
|---|---|---|---|---|---|---|---|
| Ratio using AUClast | Statistics | N = 21 | N = 19 | N = 19 | N = 17 | N = 18 | N = 21 |
| Hydromorphone/ | n | 21 | 19 | 19 | 17 | 18 | 3 |
| Hydrocodone | Mean | 0.000 | 0.001 | 0.002 | 0.003 | 0.001 | 0.000 |
| | Std. Dev. | 0.0009 | 0.0038 | 0.0027 | 0.0050 | 0.0012 | 0.0000 |
| | Median | 0.000 | 0.000 | 0.001 | 0.002 | 0.000 | 0.000 |
| | Min/Max | 0.00/0.00 | 0.00/0.02 | 0.00/0.01 | 0.00/0.02 | 0.00/0.00 | 0.00/0.00 |
| Nonhydrocodone/ | n | 21 | 19 | 19 | 17 | 18 | 3 |
| Hydrocodone | Mean | 0.366 | 0.360 | 0.327 | 0.362 | 0.448 | 0.000 |
| | Std. Dev. | 0.1189 | 0.1215 | 0.1243 | 0.1310 | 0.2144 | 0.0000 |
| | Median | 0.368 | 0.324 | 0.297 | 0.334 | 0.400 | 0.000 |
| | Min/Max | 0.11/0.81 | 0.17/0.58 | 0.20/0.76 | 0.23/0.74 | 0.22/0.84 | 0.00/0.00 |

Hydrocodone Simulations

Studies of hydrocodone formulations of the present invention were conducted to simulate the profiles associated with twice-daily administration hydrocodone for both single dose and steady state. The target doses were 10, 20, 40 and 80 mg, and the targeted minimum concentration was 5-10 ng/ml. The formulations of the study were two-component dosage forms comprising an immediate release component and a modified release component in which the hydrocodone was allocated evenly (50/50) or unevenly (20/80) across the two components. Non-compartmental parameters were used to find estimates of the unit input response and a one-compartment model was assumed for all simulations.

Non-compartmental parameters following a 10 mg oral dose of hydrocodone administered to five adult males are reported as shown in Table 12 below.

TABLE 12
Non-Compartmental Parameters

| | | |
|---|---|---|
| Cₘₐₓ | 23.6 ± 5.2 | ng/ml |
| Tₘₐₓ | 1.3 ± 0.3 | hours |
| T_{half} | 3.8 ± 0.3 | hours |

K10 and V/f were estimated to be 0.18 and 334.29 L respectively. For the absorption rate constant k01, several profiles were simulated using different estimates of k01. The secondary parameters estimates were compared to identify an appropriate ka as set forth in Table 13 below.

TABLE 13
Comparison of Absorption Rate Constant (ka).

| | | |
|---|---|---|
| ka = 1 | AUC | 166.19 |
| ka = 1 | K01-HL | 0.69 |
| ka = 1 | K10-HL | 3.85 |
| ka = 1 | CL/F | 60.17 |
| ka = 1 | Tₘₐₓ | 2.09 |
| ka = 1 | Cₘₐₓ | 20.53 |
| ka = 2 | AUC | 166.19 |
| ka = 2 | K01-HL | 0.35 |
| ka = 2 | K10-HL | 3.85 |
| ka = 2 | CL/F | 60.17 |
| ka = 2 | Tₘₐₓ | 1.32 |
| ka = 2 | Cₘₐₓ | 23.57 |
| ka = 6 | AUC | 166.19 |
| ka = 6 | K01-HL | 0.12 |
| ka = 6 | K10-HL | 3.85 |
| ka = 6 | CL/F | 60.17 |
| ka = 6 | Tₘₐₓ | 0.60 |
| ka = 6 | Cₘₐₓ | 26.84 |

ka=2 appeared to be the best estimate of the absorption rate of the instant release hydrocodone given that the maximum concentration observed and the time to maximum concentration were comparable to previous data set forth above.

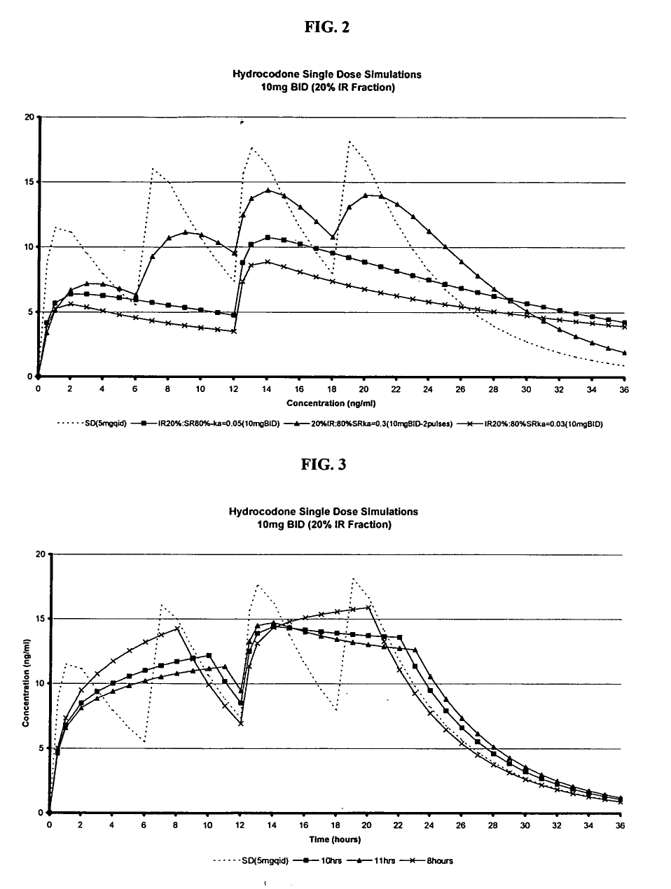

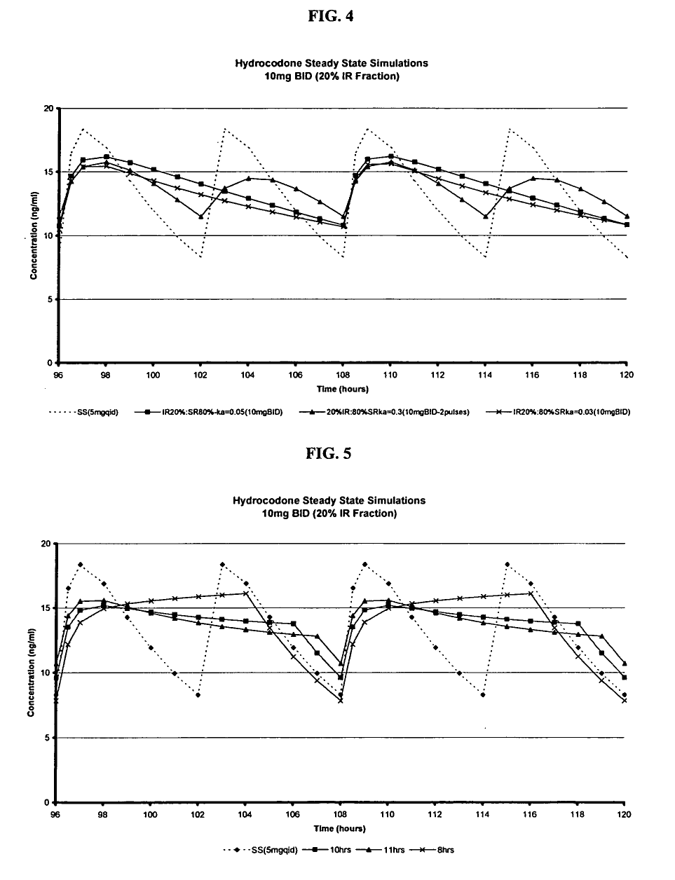

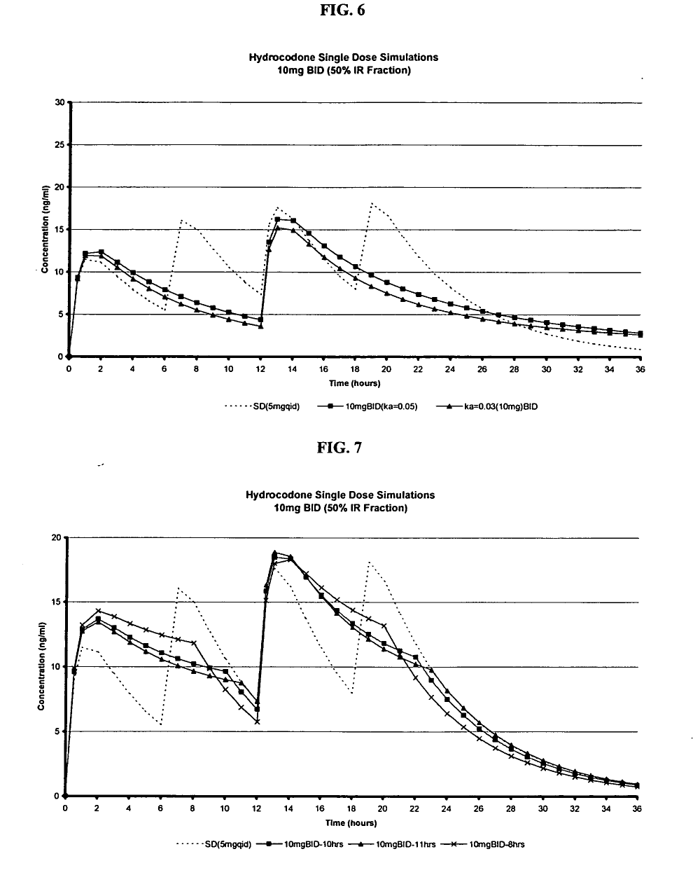

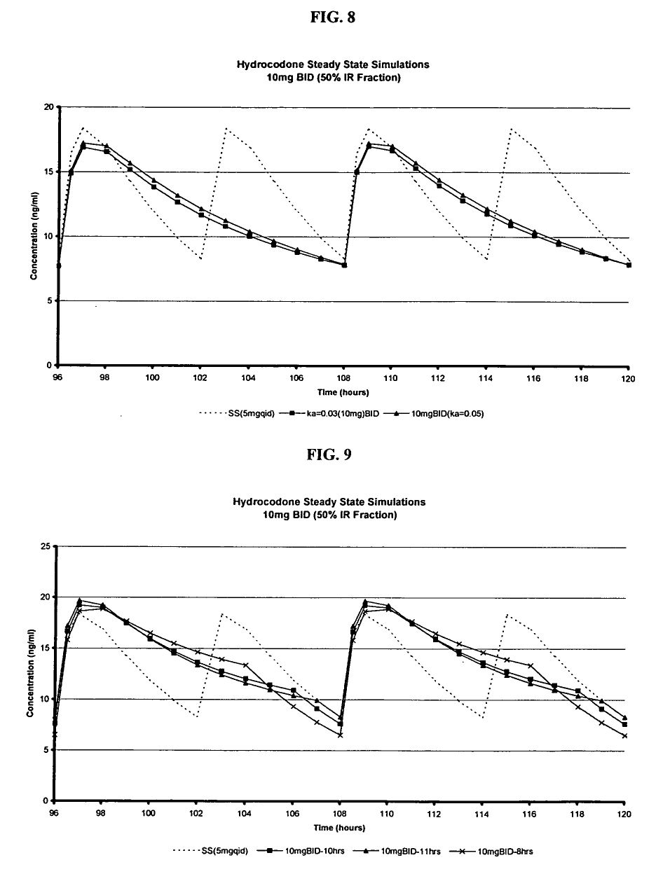

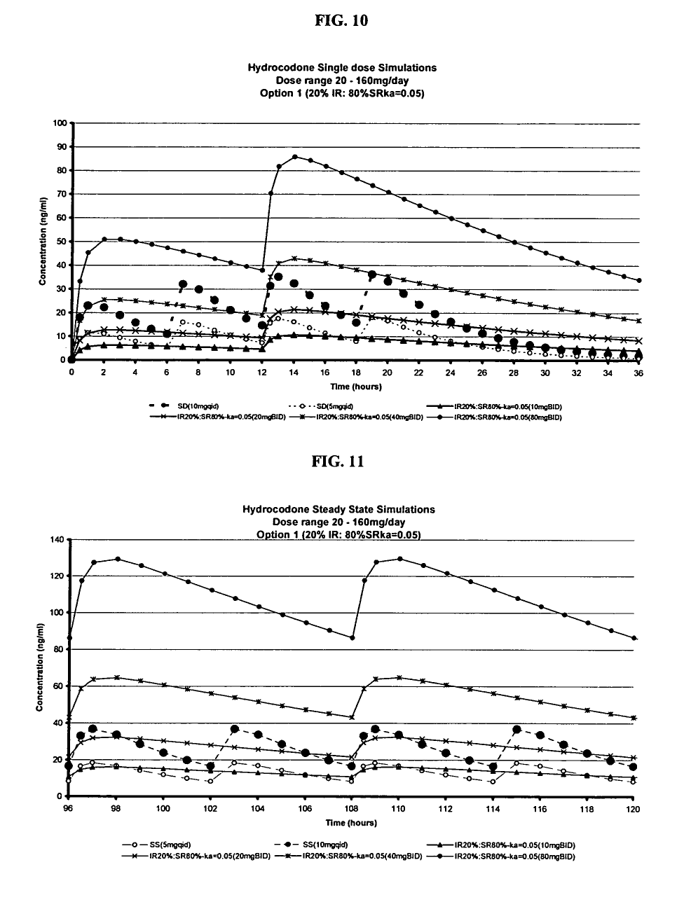

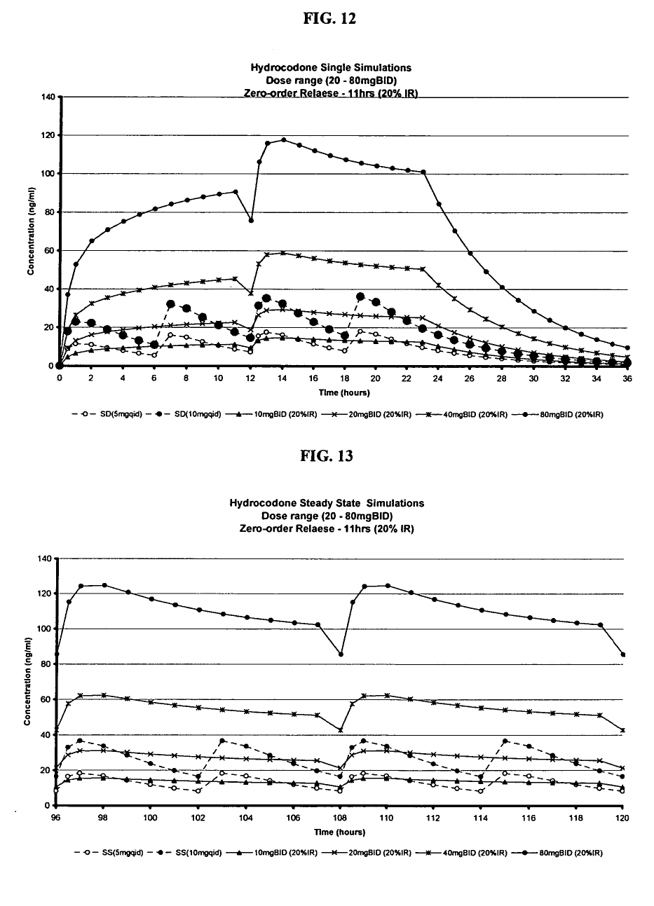

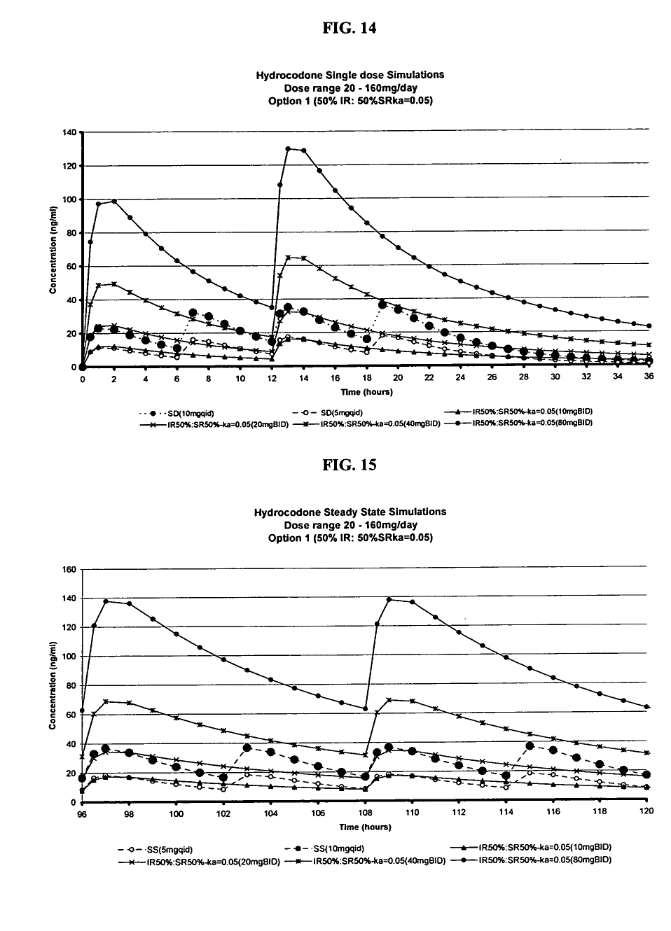

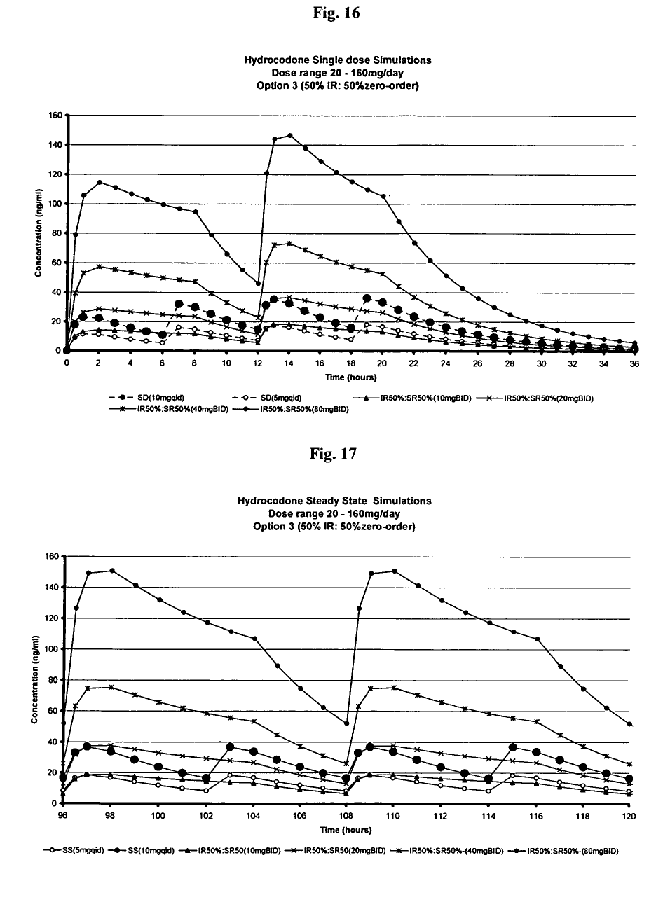

In conducting these simulations, three options were identified. Options 1 and 2 assumed a first order release and option 3 a zero-order release. Plots of the plasma concentrations of these simulations are shown in FIGS. 2 to 17.

The present invention is not limited in scope by the specific embodiments described herein. Modifications of the invention in addition to those described herein will become apparent to those skilled in the art from the foregoing description and the following claims.

What is claimed is:

1. A multiparticulate modified release composition containing at least one active ingredient and having a first component comprising a first population of active ingredient-containing particles and at least one subsequent component, each subsequent component comprising a subsequent population of active ingredient-containing particles, wherein the at least one subsequent population of active ingredient containing particles further comprises a modified release coating or, alternatively or additionally, a modified release matrix material, such that the composition following oral delivery to a subject delivers the at least one active ingredient in a bimodal or multimodal manner.

2. The multiparticulate modified release composition according to claim 1, wherein the composition comprises a first component and one subsequent component.

3. The multiparticulate modified release composition according to claim 2, wherein the first component is an immediate release component and the subsequent component is a modified release component.

4. The multiparticulate modified release composition according to claim 3, wherein the modified release component comprises particles having a modified release coating.

5. The multiparticulate modified release composition according to claim 3, wherein the modified release component comprises a modified release matrix material.

6. The multiparticulate modified release composition according to claim 1, wherein the first population of active ingredient-containing particles and the at least one subsequent population of active ingredient-containing particles comprise the same active ingredient.

7. The multiparticulate modified release composition according to claim 1, wherein the first population of active ingredient-containing particles and the at least one subsequent population of active ingredient-containing particles comprise different active ingredients.

8. The multiparticulate modified release composition according to claim 1, wherein the first population of active ingredient-containing particles contains two or more active ingredients.

9. The multiparticulate modified release composition according to claim 1, wherein the at least one subsequent population of active ingredient-containing particles contains two or more active ingredients.

10. The multiparticulate modified release composition according to claim 1, wherein the active ingredient comprises substantially one optically pure enantiomer or a mixture, racemic or otherwise, of enantiomers.

11. The multiparticulate modified release composition according to claim 1, wherein at least one of the components further comprises an enhancer.

12. The multiparticulate modified release composition according to claim 1, wherein the amount of active ingredient contained in the first and subsequent components is the same.

13. The multiparticulate modified release composition according to claim 1, wherein the amount of active ingredient contained in the first component is a minor portion of the active ingredient contained in the composition and the amount of active ingredient contained in the subsequent components is a major portion of the active ingredient contained in the composition.

14. The multiparticulate modified release composition according to claim 13, wherein the first population of active ingredient-containing particles contains from about 10% to about 40% of the active ingredient contained in the composition and the subsequent populations of active ingredient-containing particles contain from about 60% to about 90% of the active ingredient contained in the composition

15. The multiparticulate modified release composition according to claim 13, wherein the first population of active ingredient-containing particles contains about 20% of the active ingredient contained in the composition and the subsequent populations of active ingredient-containing particles contain about 80% of the active ingredient contained in the composition

16. The multiparticulate modified release composition according to claim 1, wherein the active ingredient is an opioid or a pharmaceutically acceptable salt thereof, an enantiomer thereof, or a mixture thereof.

17. The multiparticulate modified release composition according to claim 1, wherein the active ingredient is selected from the group consisting of morphine, codeine, thebaine, heroin, oxycodone, hydrocodone, dihydrocodiene, hydromorphone, oxymorphone, buprenorphine, etorphine, naloxone, nicomorphine, methadone, pethidine, fentanyl, alfentanil, sufentanil, remifentanil, carfentanyl, pentazocine, phenazocine, butorphanol, levorphanol, a pharmaceutically acceptable salt thereof, an enantiomer thereof, and a mixture thereof.

18. The multiparticulate modified release composition according to claim 1, wherein the active ingredient is morphine or a pharmaceutically acceptable salt thereof, an enantiomer thereof, or a mixture thereof.

19. The multiparticulate modified release composition according to claim 1, wherein the active ingredient is oxycodone or a pharmaceutically acceptable salt thereof, an enantiomer thereof, or a mixture thereof.

20. The multiparticulate modified release composition according to claim 1, wherein the active ingredient is hydrocodone or a pharmaceutically acceptable salt thereof, an enantiomer thereof, or a mixture thereof.

21. The multiparticulate modified release composition according to claim 1, wherein the active ingredient is hydromorphone or a pharmaceutically acceptable salt thereof, an enantiomer thereof, or a mixture thereof.

22. The multiparticulate modified release composition according to claim 1, wherein the active ingredient is an NSAID.

23. The multiparticulate modified release composition according to claim 1, wherein the active ingredient is selected from the group consisting of celecoxib, etoricoxib, rofecoxib, valdecoxib, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, tiaprofenic acid, acetylsalicylic acid, choline magnesium salicylate, choline salicylate, magnesium salicylate, sodium salicylate, a pharmaceutically acceptable salt thereof, an enantiomer thereof, and a mixture thereof.

24. The multiparticulate modified release composition according to claim 1, wherein the active ingredient is acetaminophen or a pharmaceutically acceptable salt thereof, an enantiomer thereof, or a mixture thereof.

25. The multiparticulate modified release composition according to claim 1, wherein the active ingredient is ketoprofen or a pharmaceutically acceptable salt thereof, an enantiomer thereof, or a mixture thereof.

26. The multiparticulate modified release composition according to claim 1, wherein the active ingredient is meloxicam or a pharmaceutically acceptable salt thereof, an enantiomer thereof, or a mixture thereof.

27. The multiparticulate modified release composition according to claim 1, wherein the active ingredient is naproxen or a pharmaceutically acceptable salt thereof, an enantiomer thereof, or a mixture thereof.

28. The multiparticulate modified release composition according to claim 1, wherein the active ingredients are morphine and acetaminophen, or pharmaceutically acceptable salts thereof, enantiomers thereof, or mixtures thereof.

29. The multiparticulate modified release composition according to claim 1, wherein the active ingredients are morphine and meloxicam, or pharmaceutically acceptable salts thereof, enantiomers thereof, or mixtures thereof.

30. The multiparticulate modified release composition according to claim 1, wherein the active ingredients are morphine and ketoprofen, or pharmaceutically acceptable salts thereof, enantiomers thereof, or mixtures thereof.

31. The multiparticulate modified release composition according to claim 1, wherein the active ingredients are morphine and naproxen, or pharmaceutically acceptable salts thereof, enantiomers thereof, or mixtures thereof.

32. The multiparticulate modified release composition according to claim 1, wherein the active ingredients are oxycodone and acetaminophen, or pharmaceutically acceptable salts thereof, enantiomers thereof, or mixtures thereof.

33. The multiparticulate modified release composition according to claim 1, wherein the active ingredients are oxycodone and meloxicam, or pharmaceutically acceptable salts thereof, enantiomers thereof, or mixtures thereof.

34. The multiparticulate modified release composition according to claim 1, wherein the active ingredients are oxycodone and ketoprofen, or pharmaceutically acceptable salts thereof, enantiomers thereof, or mixtures thereof.

35. The multiparticulate modified release composition according to claim 1, wherein the active ingredients are oxycodone and naproxen, or pharmaceutically acceptable salts thereof, enantiomers thereof, or mixtures thereof.

36. The multiparticulate modified release composition according to claim 1, wherein the active ingredients are hydrocodone and acetaminophen, or pharmaceutically acceptable salts thereof, enantiomers thereof, or mixtures thereof.

37. The multiparticulate modified release composition according to claim 1, wherein the active ingredients are hydrocodone and meloxicam, or pharmaceutically acceptable salts thereof, enantiomers thereof, or mixtures thereof.

38. The multiparticulate modified release composition according to claim 1, wherein the active ingredients are hydrocodone and ketoprofen, or pharmaceutically acceptable salts thereof, enantiomers thereof, or mixtures thereof.

39. The multiparticulate modified release composition according to claim 1, wherein the active ingredients are hydrocodone and naproxen, or pharmaceutically acceptable salts thereof, enantiomers thereof, or mixtures thereof.

40. The multiparticulate modified release composition according to claim 1, wherein the active ingredients are hydromorphone and acetaminophen, or pharmaceutically acceptable salts thereof, enantiomers thereof, or mixtures thereof.

41. The multiparticulate modified release composition according to claim 1, wherein the active ingredients are hydromorphone and meloxicam, or pharmaceutically acceptable salts thereof, enantiomers thereof, or mixtures thereof.

42. The multiparticulate modified release composition according to claim 1, wherein the active ingredients are hydromorphone and ketoprofen, or pharmaceutically acceptable salts thereof, enantiomers thereof, or mixtures thereof.

43. The multiparticulate modified release composition according to claim 1, wherein the active ingredients are hydromorphone and naproxen, or pharmaceutically acceptable salts thereof, enantiomers thereof, or mixtures thereof.

44. The multiparticulate modified release composition according to claim 1, wherein the first and subsequent populations of active ingredient-containing particles have different release profiles.

45. The multiparticulate modified release composition according to claim 1, wherein the first component is an immediate release component and the at least one subsequent component is a modified release component.

46. The multiparticulate modified release composition according to claim 45, which, upon administration to a patient, rapidly releases the active ingredient from the first population of active ingredient-containing particles and releases at least about 80% of the active ingredient from the at least one subsequent population of active ingredient-containing particles within about 12 hours.

47. The multiparticulate modified release composition according to claim 45, which, upon administration to a patient, rapidly releases the active ingredient from the first population of active ingredient-containing particles and releases at least about 80% of the active ingredient from the at least one subsequent population of active ingredient-containing particles within about 24 hours.

48. The multiparticulate modified release composition according to claim 45, which, upon administration to a patient, rapidly releases the active ingredient from the first population of active ingredient-containing particles and releases at least about 80% of the active ingredient from the at least one subsequent population of active ingredient-containing particles over about 12 hours.

49. The multiparticulate modified release composition according to claim 45, which, upon administration to a patient, rapidly releases the active ingredient from the first population of active ingredient-containing particles and releases at least about 80% of the active ingredient from the at least one subsequent population of active ingredient-containing particles over about 24 hours.

50. The multiparticulate modified release composition according to claim 45, which, upon administration to a patient, rapidly releases the active ingredient from the first population of active ingredient-containing particles and releases at least about 80% of the active ingredient from the at least one subsequent population of active ingredient-containing particles over at least about 12 hours.

51. The multiparticulate modified release composition according to claim 45, which, upon administration to a patient, rapidly releases the active ingredient from the first population of active ingredient-containing particles and releases at least about 80% of the active ingredient from the at least one subsequent population of active ingredient-containing particles over at least about 24 hours.

52. The multiparticulate modified release composition according to claim 1, wherein the release profile of the active ingredient upon administration to a patient mimics the release profile of the same active ingredient administered in the form of two or more doses of immediate release forms of the active ingredient.

53. The multiparticulate modified release composition according to claim 1, wherein the release profile of the active ingredient upon administration to a patient mimics the release profile of the same active ingredient administered in the form of two or more doses of the active ingredient in which one dose has an immediate release profile and at least one dose has a modified release profile.

54. A solid oral dosage form comprising a multiparticulate modified release composition according to claim 1.

55. The solid oral dosage form according to claim 54 comprising a blend of first and subsequent active ingredient-containing particles filled into hard gelatin or soft gelatin capsules.

56. The solid oral dosage form according to claim 54, wherein the first and subsequent components are separately and independently compressed into mini-tablets and filled into hard or soft gelatin capsules.

57. The solid oral dosage form according to claim 54, wherein the first component is compressed into the first layer of a multilayer tablet and the at least one subsequent component is compressed into a subsequent layer of the multilayer tablet.

58. The solid oral dosage form according to claim 54, wherein the first and subsequent components are incorporated in a rapidly dissolving dosage form.

59. The solid oral dosage form according to claim 58, wherein the rapidly dissolving dosage form is a fast-melt tablet dosage form.

60. The solid oral dosage form according to claim 54, wherein the active ingredient contained in the dosage form comprises from about 0.1 mg to about 1 g.

61. The solid oral dosage form according to claim 54, wherein the active ingredient contained in the dosage form comprises from about 10 mg to about 80 mg.

62. The solid oral dosage form according to claim 54, wherein the active ingredient contained in the dosage form comprises about 10 mg hydrocodone and the mean Cₘₐₓ is about 8.9 ng/mL ±20%.

63. The solid oral dosage form according to claim 54, wherein the active ingredient contained in the dosage form comprises about 10 mg hydrocodone and the Cₘₐₓ is from about 5 to about 15 ng/mL.

64. The solid oral dosage form according to claim 54, wherein the active ingredient contained in the dosage form comprises about 20 mg hydrocodone and the mean Cₘₐₓ is about 17.9 ng/mL ±20%.

65. The solid oral dosage form according to claim 54, wherein the active ingredient contained in the dosage form comprises about 20 mg hydrocodone and the Cₘₐₓ is from about 10 to about 27 ng/mL.

66. The solid oral dosage form according to claim 54, wherein the active ingredient contained in the dosage form comprises about 30 mg hydrocodone and the mean Cₘₐₓ is about 31.7 ng/mL ±20%.

67. The solid oral dosage form according to claim 54, wherein the active ingredient contained in the dosage form comprises about 30 mg hydrocodone and the Cₘₐₓ is from about 16 to about 46 ng/mL.

68. The solid oral dosage form according to claim 54, wherein the active ingredient contained in the dosage form comprises about 40 mg hydrocodone and the mean Cₘₐₓ is about 37.5 ng/mL ±20%.

69. The solid oral dosage form according to claim 54, wherein the active ingredient contained in the dosage form comprises about 40 mg hydrocodone and the Cₘₐₓ is from about 28 to about 62 ng/mL.

70. The solid oral dosage form according to claim 54, wherein the active ingredient contained in the dosage form comprises from about 10 mg to about 40 mg hydrocodone and the mean Tₘₐₓ is about 6 hours ±20%.

71. The solid oral dosage form according to claim 54, wherein the active ingredient contained in the dosage form comprises from about 10 mg to about 40 mg hydrocodone and the Tₘₐₓ is from about 4 to about 12 hours.

72. The solid oral dosage form according to claim 54, wherein the active ingredient contained in the dosage form comprises about 10 mg hydrocodone and the mean AUCₗₐₛₜ is about 109 ng*hr/mL ±20%.

73. The solid oral dosage form according to claim 54, wherein the active ingredient contained in the dosage form comprises about 10 mg hydrocodone and the AUCₗₐₛₜ is from about 73 to about 179 ng*hr/mL.

74. The solid oral dosage form according to claim 54, wherein the active ingredient contained in the dosage form comprises about 20 mg hydrocodone and the mean AUCₗₐₛₜ is about 212.9 ng*hr/mL ±20%.

75. The solid oral dosage form according to claim 54, wherein the active ingredient contained in the dosage form comprises about 20 mg hydrocodone and the AUCₗₐₛₜ is from about 130 to about 377 ng*hr/mL.

76. The solid oral dosage form according to claim 54, wherein the active ingredient contained in the dosage form comprises about 30 mg hydrocodone and the mean AUCₗₐₛₜ is about 392.5 ng*hr/mL ±20%.

77. The solid oral dosage form according to claim 54, wherein the active ingredient contained in the dosage form comprises about 30 mg hydrocodone and the AUCₗₐₛₜ is from about 177 to about 671 ng*hr/mL.

78. The solid oral dosage form according to claim 54, wherein the active ingredient contained in the dosage form comprises about 40 mg hydrocodone and the mean AUCₗₐₛₜ is about 464.6 ng*hr/mL ±20%.

79. The solid oral dosage form according to claim 54, wherein the active ingredient contained in the dosage form comprises about 40 mg hydrocodone and the AUCₗₐₛₜ is from about 321 to about 712 ng*hr/mL.

80. A method for the treatment of pain comprising administering a therapeutically effective amount of a composition according to claim 16.

81. A method for the treatment of pain comprising administering a therapeutically effective amount of a composition according to claim 17.

82. The method of claim 80, wherein the amount of active ingredient contained in the first component is a minor portion of the active ingredient contained in the composition and the amount of active ingredient contained in the subsequent components is a major portion of the active ingredient contained in the composition.

83. The method of claim 82, wherein the first population of active ingredient-containing particles contains about 20% of the active ingredient contained in the composition and the subsequent populations of active ingredient-containing particles contain about 80% of the active ingredient contained in the composition.
